(19) United States
(12) Patent Application Publication
(10) Pub. No.: US 2024/0382632 A1
(43) Pub. Date: Nov. 21, 2024

(54) METHODS OF PREPARATION OF 18F LABELLED SILYL-FLUORIDE COMPOUNDS

(71) Applicant: Blue Earth Diagnostics Limited, Oxford (GB)

(72) Inventors: Romain BEJOT, Oxford (GB); Nigel OSBORN, Oxford (GB); Alexander Josef WURZER, Oxford (GB)

(21) Appl. No.: 18/694,567

(22) PCT Filed: Sep. 26, 2022
(86) PCT No.: PCT/GB2022/052432

§ 371 Date: Mar. 22, 2024

(30) Foreign Application Priority Data
Sep. 24, 2021 (GB) 2113646.0

(51) Int. Cl. A61K 51/04; C07B 59/00

(52) U.S. Cl. CPC A61K 51/0497; A61K 51/0482; C07B 59/002; A61K 2123/00; C07B 2200/05

(57) ABSTRACT

The present invention relates to methods of preparation of a solution comprising an ¹⁸F labelled silyl-fluoride compound. Compounds and compositions obtained by the methods of the invention may be useful as positron emission tomography (PET) imaging agents. Compounds and compositions obtained by the methods of the invention may be useful in the diagnosis or imaging of angiogenesis or cancer.

The present invention relates to methods of preparation of a solution comprising an ¹⁸F labelled silyl-fluoride compound. Compounds and compositions obtained by the methods of the invention may be useful as positron emission tomography (PET) imaging agents. Compounds and compositions obtained by the methods of the invention may be useful in the diagnosis or imaging of angiogenesis or cancer.

### BACKGROUND OF THE INVENTION

### Prostate Cancer

Prostate Cancer (PCa) remained over the last decades the most common malignant disease in men with high incidence for poor survival rates. Due to its overexpression in prostate cancer, prostate-specific membrane antigen (PSMA) or glutamate carboxypeptidase II (GCP II) proved its eligibility as excellent target for the development of highly sensitive radiolabelled agents for endoradiotherapy and imaging of PCa. Prostate-specific membrane antigen is an extracellular hydrolase whose catalytic center comprises two zinc (II) ions with a bridging hydroxido ligand. It is highly upregulated in metastatic and hormone-refractory prostate carcinomas, but its physiologic expression has also been reported in kidneys, salivary glands, small intestine, brain and, to a low extent, also in healthy prostate tissue. In the intestine, PSMA facilitates absorption of folate by conversion of pteroylpoly-γ-glutamate to pteroylglutamate (folate). In the brain, it hydrolyses N-acetyl-L-aspartyl-L-glutamate (NAAG) to N-acetyl-L-aspartate and glutamate.

### Prostate-Specific Membrane Antigen (PSMA)

Prostate-specific membrane antigen (PSMA) is a type II transmembrane glycoprotein that is highly overexpressed on prostate cancer epithelial cells. Despite its name, PSMA is also expressed, to varying degrees, in the neovasculature of a wide variety of nonprostate cancers. Among the most common nonprostate cancers to demonstrate PSMA expression include breast, lung, colorectal, and renal cell carcinoma.

The general necessary structures of PSMA targeting molecules comprise a binding unit that encompasses a zinc-binding group (such as urea, phosphinate or phosphoramidate) connected to a P1′ glutamate moiety, which warrants high affinity and specificity to PSMA and is typically further connected to an effector functionality. The effector part is more flexible and to some extent tolerant towards structural modifications. The entrance tunnel accommodates two other prominent structural features, which are important for ligand binding. The first one is an arginine patch, a positively charged area at the wall of the entrance funnel and the mechanistic explanation for the preference of negatively charged functionalities at the P1 position of PSMA. This appears to be the reason for the preferable incorporation of negative charged residues within the ligand-scaffold. An in-depth analysis about the effect of positive charges on PSMA ligands has been, to our knowledge, so far not conducted. Upon binding, the concerted repositioning of the arginine side chains can lead to the opening of an S1 hydrophobic accessory pocket, the second important structure that has been shown to accommodate an iodo-benzyl group of several urea based inhibitors, thus contributing to their high affinity for PSMA.

Zhang et al. discovered a remote binding site of PSMA, which can be employed for bidentate binding mode (Zhang et al., Journal of the American Chemical Society 132, 12711-12716 (2010)). The so called arene-binding site is a simple structural motif shaped by the side chains of Arg463, Arg511 and Trp541, and is part of the GCPII entrance lid. The engagement of the arene binding site by a distal inhibitor moiety can result in a substantial increase in the inhibitor affinity for PSMA due to avidity effects. PSMA I&T was developed with the intention to interact this way with PSMA, albeit no crystal structure analysis of binding mode is available. A necessary feature according to Zhang et al. is a linker unit (Suberic acid in the case of PSMA I&T) which facilitates an open conformation of the entrance lid of GCPII and thereby enabling the accessibility of the arene-binding site. It was further shown that the structural composition of the linker has a significant impact on the tumor-targeting and biologic activity as well as on imaging contrast and pharmacokinetics (Liu et al., Bioorganic & medicinal chemistry letters 21, 7013-7016 (2011)), properties which are crucial for both high imaging quality and efficient targeted endoradiotherapy.

Two categories of PSMA targeting inhibitors are currently used in clinical settings. On the one side there are tracers with chelating units for radionuclide complexation such as PSMA I&T or related compounds. On the other side there are small molecules, comprising a targeting unit and effector molecules.

### ¹⁸F-rhPSMA-7 and ¹⁸F-rhPSMA-7.3

The concept of radiohybrid PSMA ligands (rhPSMA) has been introduced for the first time in 2017 at the Klinikum rechts der Isar in Munich (Wurzer et al., J. Nucl. Med. 2020;61 (5): 735-42). rhPSMA ligands offer two binding sites for labeling with radionuclides, i.e. ¹⁸F , ⁶⁸Ga, ¹⁷⁷Lu or their nonradioactive counterparts within one molecule. The structural formula consists of a Silicon-Fluoride Acceptor (SiFA) for ¹⁸F-labeling in an isotopic exchange reaction, a DOTA-GA-chelator for (radio-) metal complexation and the Glu-Urea-Glu-(EuE-)-based inhibitor group for binding to the enzymatic pocket of the prostate specific membrane antigen (PSMA) (Wurzer et al., J. Nucl. Med. 2020;61 (5): 735-42). In the case of ¹⁸F-rhPSMA-7, the DOTA-GA (2-(4,7, 10-tris (carboxymethyl)-1,4,7, 10-tetraazacyclododecan-1-yl) pentanedioic acid)) chelator complexes nonradioactive nat Ga, whereas the cold ¹⁹F of the SiFA-group is substituted by ¹⁸F during the labeling process. This ¹⁸F-labeled PET tracer allows fast synthesis, longer half-life compared with ⁶⁸Ga-PSMA-11, larger-scale production, lower positron range and is therefore increasing interest in its use in the imaging of patients with prostate cancer (Eiber et al., J. Nucl. Med. 2020;61 (5): 696-701; Oh et al., J. Nucl. Med. 2020;61 (5): 702-9).

Other agents used for selective PSMA imaging include PSMA HBED-CC, PSMA-617 and PSMA I&T, which are predominantly labelled with ⁶⁸Ga (88.9% β⁺, E_{β+, max}=1.89 MeV, t_{1/2}=68 min). Among these ⁶⁸Ga-PSMA-HBED-CC (also known as ⁶⁸Ga-PSMA-11), is preferred for PET imaging of PCa.

### ¹⁸F Labelling

Several groups have focused on the development of novel ¹⁸F-labelled urea-based inhibitors for PCa diagnosis. In contrast to the radiometal ⁶⁸Ga, which can be obtained from commercially distributed ⁶⁸Ge/⁶⁸Ga radionuclide generators (⁶⁸Ge; t_{1/2}=270.8 d), the radioisotope ¹⁸F-fluoride (96.7% β⁺, E_{β+, max}=634 keV) requires an on-site cyclotron for its production. Despite this limitation, ¹⁸F offers due to its longer half-live (t_{1/2}=109.8 min) and its lower positron energy, significant advantages in terms of routine-handling and image quality. Additionally, there is the possibility for largescale production in a cyclotron, which would be beneficial for a higher patient throughput and reduction of production costs. The ¹⁸F-labelled urea-based PSMA inhibitor ¹⁸F-DCFPyl demonstrated promising results in the detection of primary and metastatic PCa (Rowe et al., Molecular Imaging and Biology, 1-9 (2016)) and superiority to ⁶⁸Ga-PSMA-HBED-CC in a comparative study (Dietlein et al., Molecular Imaging and Biology 17, 575-584 (2015)). Based on the structure of PSMA-617, the ¹⁸F-labelled analogue PSMA-1007 was recently developed, which showed comparable tumor-to-organ ratios (Cardinale et al., Journal of nuclear medicine: official publication, Society of Nuclear Medicine 58, 425-431 (2017); Giesel et al., European journal of nuclear medicine and molecular imaging 43, 1929-1930 (2016)). A comparative study with ⁶⁸Ga-PSMA-HBED-CC revealed similar diagnostic accuracy of both tracers and a reduced urinary clearance of ¹⁸F-PSMA-1007, enabling a better assessment of the prostate (Giesel et al., European journal of nuclear medicine and molecular imaging 44, 678-688 (2017)).

An attractive approach for introducing ¹⁸F labels is the use of silicon fluoride acceptors (SIFA). Silicon fluoride acceptors are described, for example, in Lindner et al., Bioconjugate Chemistry 25, 738-749 (2014). In order to preserve the silicon-fluoride bond, the use of silicon fluoride acceptors introduces the necessity of sterically demanding groups around the silicone atom. This in turn renders silicon fluoride acceptors highly hydrophobic. In terms of binding to the target molecule, in particular to the target molecule which is PSMA, the hydrophobic moiety provided by the silicone fluoride acceptor may be exploited for the purpose of establishing interactions of the radio-diagnostic or-therapeutic compound with the hydrophobic pocket described in Zhang et al., Journal of the American Chemical Society 132, 12711-12716 (2010). Yet, prior to binding, the higher degree of lipophilicity introduced into the molecule poses a severe problem with respect to the development of radiopharmaceuticals with suitable in vivo biodistribution, i.e. low unspecific binding in non-target tissue.

Previous methods for the preparation of ¹⁸F labelled silyl-fluoride compounds, such as those described in WO2020/157177 rely on the use of oxalic acid for pH adjustment. The amount of oxalic acid used in such methods has been recognised as a crucial parameter for radiochemical yield (EJNMMI Radiopharm Chem. 2021;6 (1): 4). In methods that make use of oxalic acid residual oxalic acid remains in the product composition. Oxalic acid is currently not recognized as a substance for pharmaceutical use and may be toxic. Moreover, no suitable test method for oxalic acid is currently described in the European Pharmacopoeia.

As such there is a need for methods of preparation of ¹⁸F labelled silyl-fluoride compounds and compositions comprising such compounds with acceptable radiochemical yields which do not make use of oxalic acid.

### THE INVENTION

The invention relates to methods of preparing a solution comprising an ¹⁸F labelled silyl-fluoride compound. The methods of the invention include conversion of ¹⁹F to ¹⁸F in compounds comprising a silyl-fluoride (SIFA) moiety.

The invention provides a method of preparing a solution comprising an ¹⁸F labelled silyl-fluoride compound, wherein the method comprises:
a) passing aqueous ¹⁸F solution through an anion exchange cartridge;
b) eluting the ¹⁸F from the cartridge using a solution comprising [2.2.2]-cryptand, an inorganic base, an organic solvent and water;
c) azeotropically drying the eluent; and
d) adding to the azeotropically dried eluent a solution comprising acetic acid and a compound having a ¹⁹F silyl-fluoride bond;

wherein said solution comprising the ¹⁸F labelled silyl-fluoride compound further comprises acetic acid at a concentration of at least 100 mM.

Compounds and compositions obtained by the methods of the invention may be useful as positron emission tomography (PET) imaging agents. Compounds and compositions obtained by the methods of the invention may be useful in the diagnosis or imaging of angiogenesis or cancer. Compounds and compositions obtained by the methods of the invention may be useful in the diagnosis or imaging of neoangiogenesis/angiogenesis or cancer wherein the cancer is prostate, breast, lung, colorectal or renal cell carcinoma. In particular, compounds and compositions obtained by the methods of the invention may be useful in the diagnosis or imaging of prostate cancer.

### DETAILED DESCRIPTION OF THE INVENTION

The invention relates to methods of preparing a solution comprising an ¹⁸F labelled silyl-fluoride compound. The methods of the invention include conversion of ¹⁹F to ¹⁸F in compounds comprising a silyl-fluoride (SIFA) moiety.

The invention provides a method of preparing a solution comprising an ¹⁸F labelled silyl-fluoride compound, wherein the method comprises:
a) passing aqueous ¹⁸F solution through an anion exchange cartridge;
b) eluting the ¹⁸F from the cartridge using a solution comprising [2.2.2]-cryptand, an inorganic base, an organic solvent and water;
c) azeotropically drying the eluent; and
d) adding to the azeotropically dried eluent a solution comprising acetic acid and a compound having a ¹⁹F silyl-fluoride bond;

wherein said solution comprising the ¹⁸F labelled silyl-fluoride compound further comprises acetic acid at a concentration of at least 100 mM.

In the methods described herein, the prepared solution comprising the ¹⁸F labelled silyl-fluoride compound may comprise acetic acid at a concentration of at least 100 mM. The prepared solution comprising the ¹⁸F labelled silyl-fluoride compound may comprise acetic acid at a concentration of at least 130 mM. The prepared solution comprising the ¹⁸F labelled silyl-fluoride compound may comprise acetic acid at a concentration of at 100-200 mM. The prepared solution comprising the ¹⁸F labelled silyl-fluoride compound may comprise acetic acid at a concentration of at 130-160 mM. The concentration of acetic acid in the solution comprising an ¹⁸F labelled silyl-fluoride compound may be 100-200 mM. The concentration of acetic acid in the solution comprising an ¹⁸F labelled silyl-fluoride compound may be 130-160mM. The prepared solution comprising the ¹⁸F labelled silyl-fluoride compound may comprise acetic acid and carbonate at a molar ratio of between 2:1 and 15:1.

In the methods described herein, the prepared solution comprising the ¹⁸F labelled silyl-fluoride compound may also comprise non-acetate organic acid species at a lower concentration relative to the acetic acid. The ¹⁸F labelled silyl-fluoride compound may comprise non-acetate organic acid species at a concentration of less than 1 mM. The ¹⁸F labelled silyl-fluoride compound may comprise non-acetate organic acid species at a concentration of less than 0.5 mM. The ¹⁸F labelled silyl-fluoride compound may comprise non-acetate organic acid species at a concentration of less than 0.3 mM. The ¹⁸F labelled silyl-fluoride compound may comprise non-acetate organic acid species at a concentration of less than 0.1 mM. The non-acetate organic acid species can be any organic acid other than acetic acid or any organic anion other than acetate arising from deprotonation of the respective acid. Examples of non-acetate organic acid species that may be present at low concentration in solutions prepared using the methods herein include for example methylene butanedioate, arising from methylene butanedioic acid.

In the methods described herein, the prepared solution comprising acetic acid and a compound having a ¹⁹F silyl-fluoride bond may comprise an aprotic solvent. The aprotic solvent present in the prepared solution comprising acetic acid and a compound having a ¹⁹F silyl-fluoride bond may for example be dimethyl sulfoxide (DMSO), acetonitrile or dimethylformamide (DMF), or any combination thereof. The aprotic solvent may be DMSO. The aprotic solvent may be acetonitrile. The aprotic solvent may be DMF.

In the methods described herein, the anion exchange cartridge employed in step a) may be a quaternary methyl ammonium carbonate anion exchange cartridge. The anion exchange cartridge may for example be a Sep-Pak® Accell Plus QMA Carbonate cartridge. The anion exchange cartridge may be preconditioned with water prior to addition of aqueous ¹⁸F solution.

In the methods described herein, ¹⁸F is eluted from the cartridge in step b) using a solution comprising [2.2.2]-cryptand, an inorganic base, an organic solvent and water. The inorganic base can be potassium carbonate. The organic solvent can be acetonitrile. The [2.2.2]-cryptand may be present in the solution at a concentration of 5-60 mg/mL. The [2.2.2]-cryptand may be present in the solution at a concentration of 15-53 mg/mL. The [2.2.2]-cryptand may be present in the solution at a concentration of 10-20 mg/mL. The [2.2.2]-cryptand may be present in the solution at a concentration of 15 mg/mL. The potassium carbonate may be present in the solution at a concentration of 2-10 mg/mL. The potassium carbonate may be present in the solution at a concentration of 2 mg/mL. The solution comprising [2.2.2]-cryptand, an inorganic base, an organic solvent and water may comprise 2-10 mg/mL potassium carbonate and 15-53 mg/mL [2.2.2]-cryptand. The acetonitrile/water ratio in the solution may be between 5/1 and 20/1 (v/v). The acetonitrile/water ratio in the solution may be 9/1 (v/v). In step b) ¹⁸F fluoride becomes bound to cryptand and is thereby removed from the column with the eluent solution.

[2.2.2]-Cryptand (K222) is a well known chelating agent of formula N(CH₂CH₂OCH₂CH₂OCH₂CH₂)₃N (structure shown below) which possesses a high affinity for alkali metal cations such as potassium (K⁺).

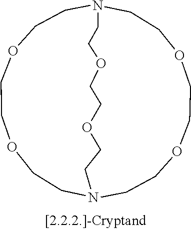 [SMILES: C1COCCN2CCOCCOCCN(CCO1)CCOCCOCC2]

In the methods described herein, azeotropic drying in step c) comprises a process whereby water is removed from the eluent by the addition of another liquid that forms an azeotrope with the water. This allows water to be removed without complete removal of organic solvent. Equipment such as a Dean-Stark apparatus, distilling trap or an equivalent device for use on smaller scales may be used to assist in the azeotropic removal of water.

In the methods described herein, the solution comprising acetic acid and a compound having a ¹⁹F silyl-fluoride bond in step d) may be prepared using a 100-200 mM solution of acetic acid in DMSO. The solution comprising acetic acid and a compound having a ¹⁹F silyl-fluoride bond in step d) may be prepared using a 160 mM solution of acetic acid in DMSO.

The solution comprising the ¹⁸F labelled silyl-fluoride compound and acetic acid is prepared according to the methods described herein is purified prior to administration as a radio-diagnostic or imaging agent. Said purification may comprise passing the solution through a solid-phase extraction cartridge containing a hydrophobic resin. The solid-phase extraction cartridge containing a hydrophobic resin may be a Sep-Pak® Plus Short tC18 cartridge. The cartridge may be preconditioned with EtOH, followed by H₂O. The solution comprising the ¹⁸F labelled silyl-fluoride compound and acetic acid may be diluted with citrate buffer (pH 5) and passed through the cartridge followed by citrate buffer. The product may be eluted from the cartridge using EtOH/water mixture.

Provided is a method of preparing a solution comprising an ¹⁸F labelled silyl-fluoride compound, wherein the method comprises:
a) passing aqueous ¹⁸F solution through an anion exchange cartridge;
b) eluting the ¹⁸F from the cartridge using a solution comprising [2.2.2]-cryptand, potassium carbonate, acetonitrile and water;
c) azeotropically drying the eluent; and
d) adding to the azeotropically dried eluent a solution comprising acetic acid and a compound having a ¹⁹F silyl-fluoride bond;

wherein said solution comprising the ¹⁸F labelled silyl-fluoride compound further comprises acetic acid at a concentration of 100-200 mM.

Provided is a method of preparing a solution comprising an ¹⁸F labelled silyl-fluoride compound, wherein the method comprises:
a) passing aqueous ¹⁸F solution through an anion exchange cartridge;
b) eluting the ¹⁸F from the cartridge using a solution comprising [2.2.2]-cryptand, an inorganic base, an organic solvent and water;
c) azeotropically drying the eluent;
d) adding to the azeotropically dried eluent a solution comprising acetic acid and a compound having a ¹⁹F silyl-fluoride bond; and
e) diluting the resultant solution comprising the ¹⁸F labelled silyl-fluoride compound and acetic acid at a concentration of at least 100 mM with citrate buffer and passing it through a solid-phase extraction cartridge containing a hydrophobic resin.

The methods described herein require ‘inverse addition’ of acidified ¹⁹F silyl-fluoride precursor solution to alkaline [¹⁸F] fluoride/K222 instead of addition of alkaline [¹⁸F] fluoride/K222 to the acidic precursor solution. As shown in FIG. 1, a higher amount of acid is required to prevent the isomerisation of the ¹⁹F silyl-fluoride precursor and ¹⁸F labelled silyl-fluoride compound in the presence of carbonate. The quantity and nature of acid employed is therefore a crucial aspect of the invention. The use of the azeotropic drying process with ‘inverse addition’ of solutions required optimisation of acetic acid content in order to minimise isomerisation under basic conditions.

The invention also provides compositions prepared according to the methods described herein. Accordingly the invention provides a liquid composition comprising an ¹⁸F labelled silyl-fluoride compound, acetic acid at a concentration of 100-200 mM and no non-acetate organic acid species at a concentration of 0.3 mM or higher. The invention provides a liquid composition comprising an ¹⁸F labelled silyl-fluoride compound, acetic acid at a concentration of 130-160 mM and no non-acetate organic acid species at a concentration of 0.3 mM or higher.

The invention provides a liquid composition comprising an ¹⁸F labelled silyl-fluoride compound of formula (IIIa), (1a), (1b), (1c), (3a), (3b), (3c) or (5a) as described below, acetic acidat a concentration of 100-200 mM and no non-acetate organic acid species at a concentration of 0.3 mM or higher. The invention provides a liquid composition comprising an ¹⁸F labelled silyl-fluoride compound of formula (IIIa), (1a), (1b), (1c), (3a), (3b), (3c) or (5a) as described below, acetic acid at a concentration of 130-160 mM and no non-acetate organic acid species at a concentration of 0.3 mM or higher.

In the methods described herein, silyl-fluoride as in “¹⁸F labelled silyl-fluoride compound” or “compound having a ¹⁹F silyl-fluoride bond” refers to any moiety possessing a covalent bond between Si and F. The silyl-fluoride (SIFA) moiety may have the structure represented by formula (10a):

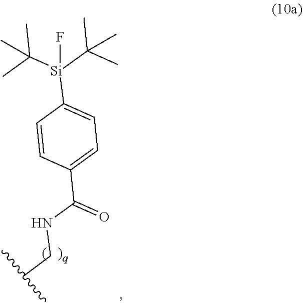

wherein q is 0 to 3.

The silyl-fluoride (SIFA) moiety may have the structure represented by formula (10b):

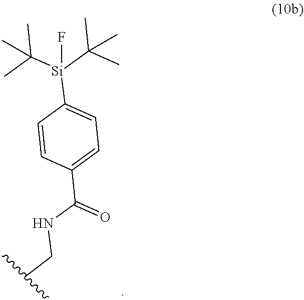 [SMILES: *CNC(=O)c1ccc([Si](F)(C(C)(C)C)C(C)(C)C)cc1]

In the compounds and moieties represented structurally herein F is to be understood to encompass both ¹⁹F and ¹⁸F.

In the methods described herein, the ^{18/19}F labelled silyl-fluoride compound may be a compound of formula (IIIa):

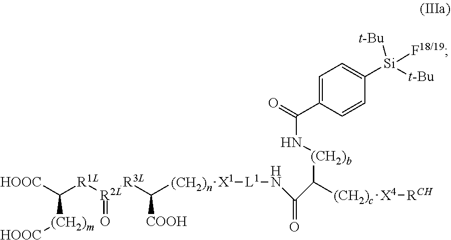

or a pharmaceutically acceptable salt thereof, wherein:
m is an integer of 2 to 6;
n is an integer of 2 to 6;
b is an integer of 0 to 4;
c is an integer of 0 to 4;
R^{1L} is CH2, NH or O;
R^{3L} is CH2, NH or O;
R^{2L} is C or P (OH);
X¹ is selected from an amide bond, an ether bond, a thioether bond, an ester bond, a thioester bond, an urea bridge, and an amine bond;
L¹ is a divalent linking group with a structure selected from an oligoamide, an oligoether, an oligothioether, an oligoester, an oligothioester, an oligourea, an oligo (ether-amide), an oligo (thioether-amide), an oligo (ester-amide), an oligo (thioester-amide), oligo (urea-amide), an oligo (ether-thioether), an oligo (ether-ester), an oligo (ether-thioester), an oligo ether-urea), an oligo (thioether-ester), an oligo (thioether-thioester), an oligo (thioether-urea), an oligo (ester-thioester), an oligo (ester-urea), and an oligo (thioester-urea), wherein L¹ is optionally substituted with one or more substitutents independently selected from —OH, —OCH₃, —COOH, —COOCH₃, —NH₂, and —NHC(NH)NH₂;

X⁴ is selected from an amide bond, an ether bond, a thioether bond, an ester bond, a thioester bond, a urea bridge, an amine bond, a linking group of the formula:

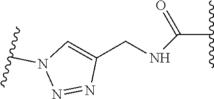 [SMILES: *C(=O)NCc1cn(*)nn1]

wherein the amide bond marked by [symbol] is formed with the chelating group, and a linking group of the formula:

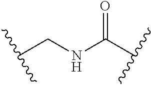 [SMILES: *CNC(*)=O]

wherein the bond marked by [symbol] at the carbonyl end is formed with the chelating group; and

R^{CH} is chelating group optionally containing a chelated radioactive or nonradioactive cation.

In the methods described herein, the ¹⁸F labelled silyl-fluoride compound may be a compound of formula (1a):

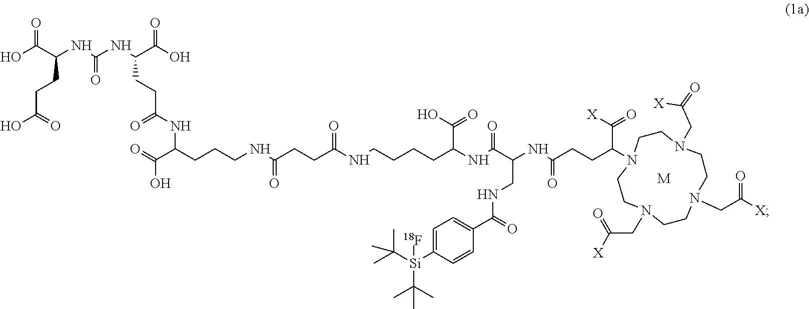

and the compound having a ¹⁹F silyl-fluoride bond may be a compound of formula (2a):

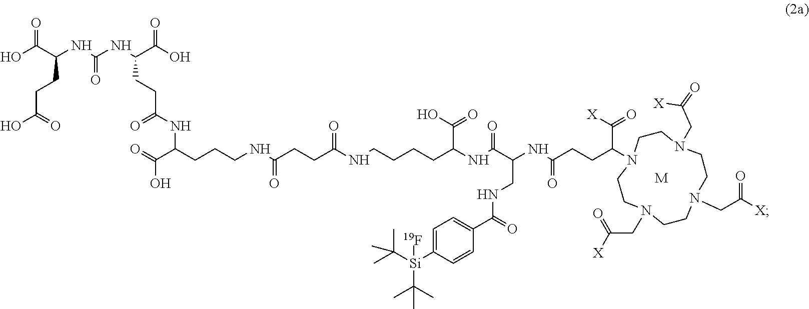

wherein each X is independently OH or O⁻; and

M is either a chelated cation or is absent.

In the methods described herein, the ¹⁸F labelled silyl-fluoride compound may be a compound of formula (1b):

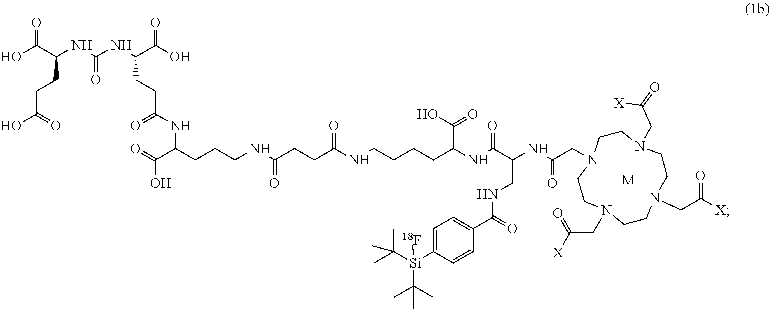

and the compound having a ¹⁹F silyl-fluoride bond may be a compound of formula (2b):

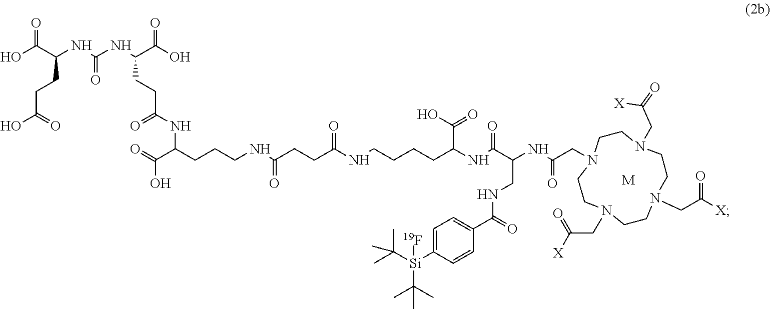

wherein each X is independently OH or O⁻; and

M is either a chelated cation or is absent.

In the methods described herein, the ¹⁸F labelled silyl-fluoride compound may be a compound of formula (1c):

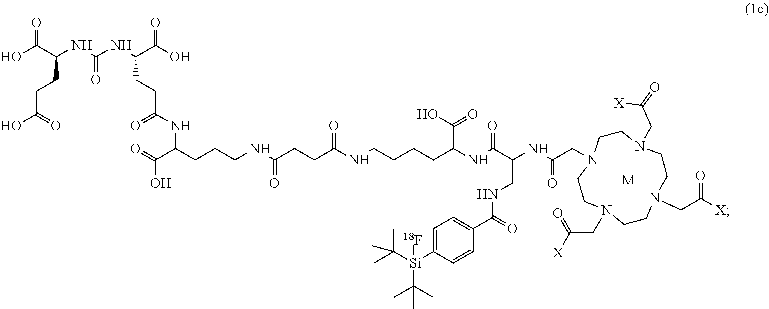

and the compound having a ¹⁹F silyl-fluoride bond may be a compound of formula (2c):

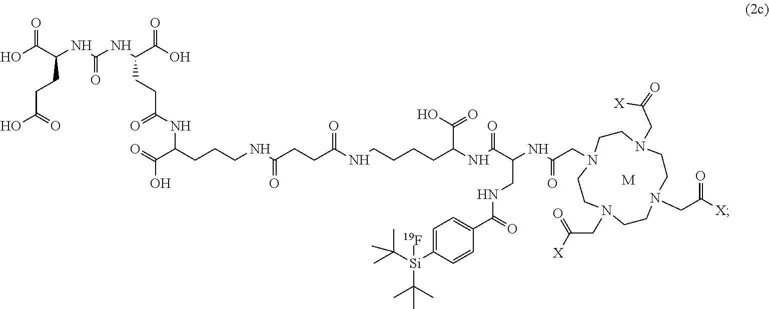

wherein each X is independently OH or O⁻; and

M is either a chelated cation or is absent.

In the methods described herein, the ¹⁸F labelled silyl-fluoride compound may be a compound of formula (3):

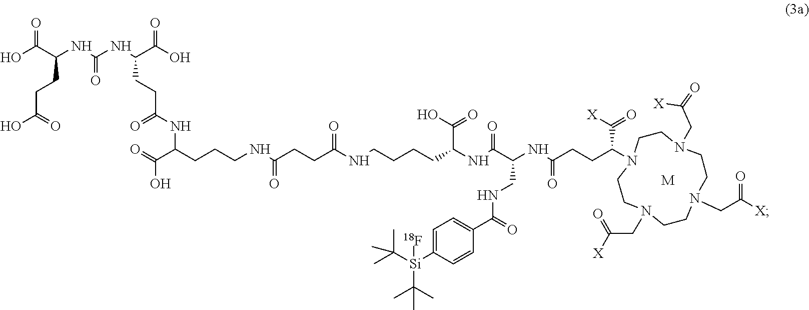

and the compound having a ¹⁹F silyl-fluoride bond may be a compound of formula (4a):

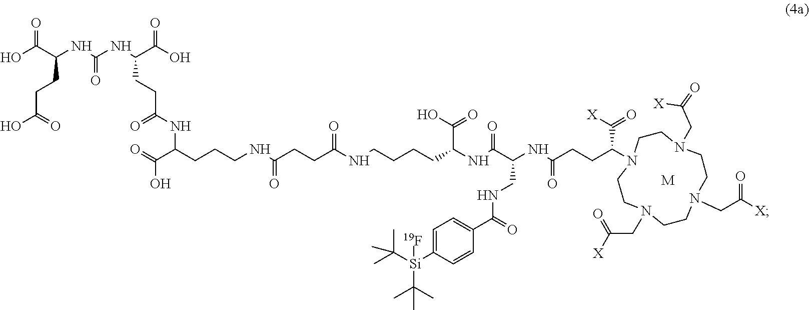

wherein each X is independently OH or O⁻; and

M is either a chelated non-radioactive cation or is absent.

In the methods described herein, the ¹⁸F labelled silyl-fluoride compound may be a compound of formula (3b):

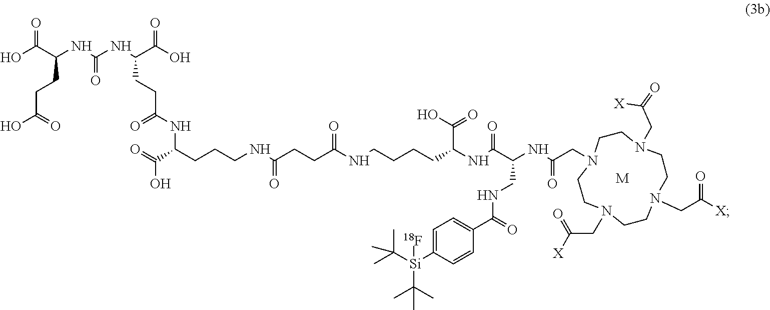

and the compound having a ¹⁹F silyl-fluoride bond may be a compound of formula (4b):

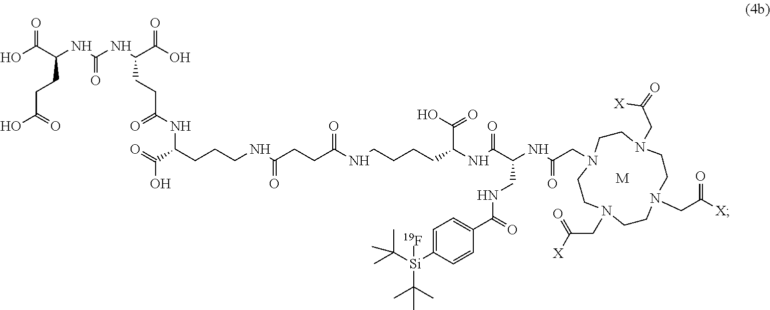

wherein each X is independently OH or O⁻; and

M is either a chelated non-radioactive cation or is absent.

In the methods described herein, the ¹⁸F labelled silyl-fluoride compound may be a compound of formula (3c):

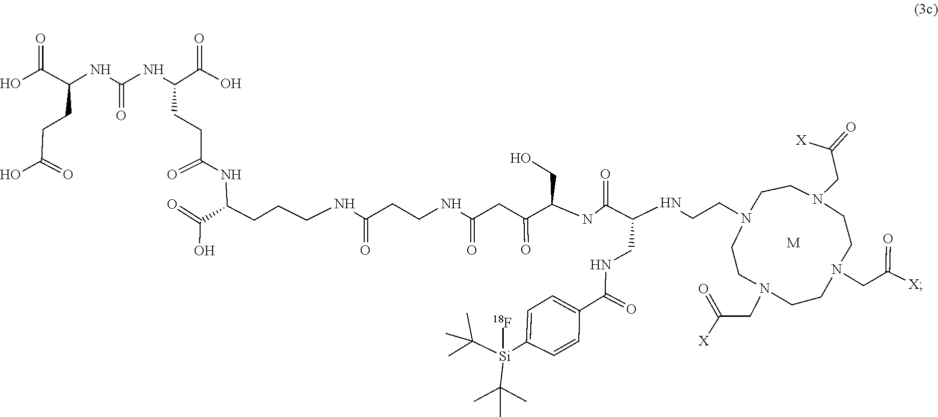

and the compound having a ¹⁹F silyl-fluoride bond may be a compound of formula (4c):

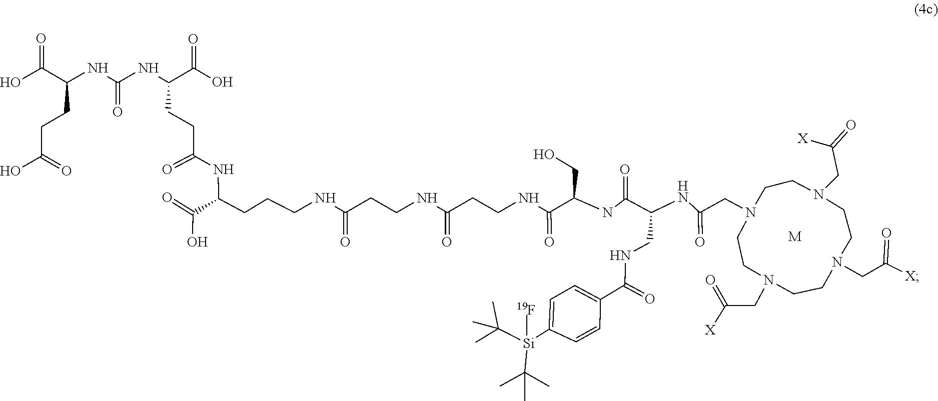

wherein each X is independently OH or O⁻; and

M is either a chelated non-radioactive cation or is absent.

In the compounds of formula (1a) to (4c) described above, M can be selected from the cations of Sc, Cu, Ga, Y, In, Tb, Ho, Lu, Re, Pb, Bi, Ac, Er and Th. M can be Ga³⁺.

In the methods described herein, the ¹⁸F labelled silyl-fluoride compound may be a compound of formula (5a):

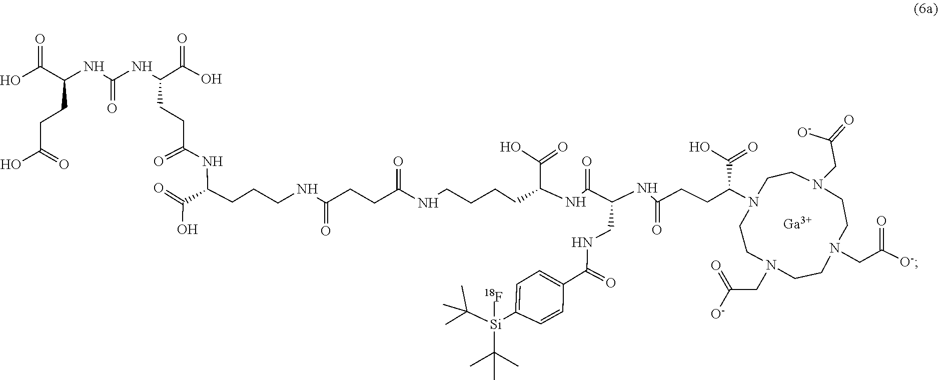

and the compound having a ¹⁹F silyl-fluoride bond may be a compound of formula (6a):

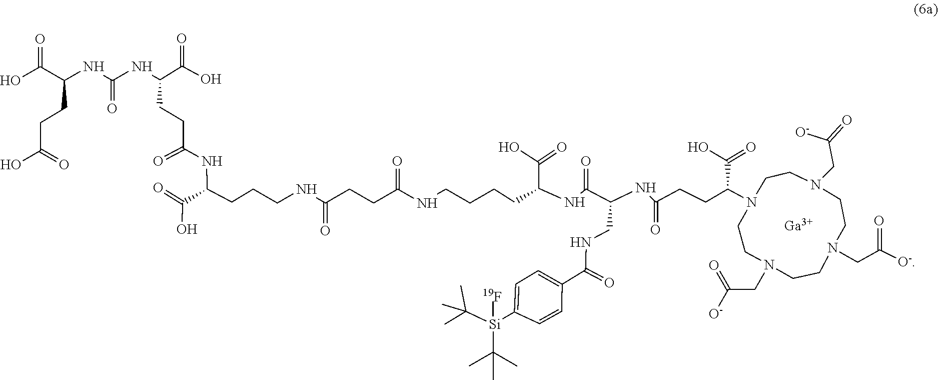

Compositions prepared according to the methods of the invention may comprise any one of the ¹⁸F-labelled compounds of formula (IIIa), (1a), (1b), (1c), (3a), (3b), (3c) or (5a) as described above.

Compounds and compositions obtained by the methods of the invention may be useful as positron emission tomography (PET) imaging agents. Compounds and compositions obtained by the methods of the invention may be useful in the diagnosis or imaging of angiogenesis or cancer. Compounds and compositions obtained by the methods of the invention may be useful in the diagnosis or imaging of neoangiogenesis/angiogenesis or cancer wherein the cancer is prostate, breast, lung, colorectal or renal cell carcinoma. In particular, compounds and compositions obtained by the methods of the invention may be useful in the diagnosis or imaging of prostate cancer.

### EXAMPLES

### rhPMSA-7, rhPMSA-10 & 2C013

Synthesis protocols for the ¹⁹F compounds ¹⁹F-rhPSMA-7.1, ¹⁹F-rhPSMA-7.2, ¹⁹F-rhPSMA-7.3, ¹⁹F-rhPSMA-7.4, ¹⁹F-rhPSMA-10.1, ¹⁹F-rhPSMA-10.2 and ¹⁹F-2C013 (shown below) are provided in WO2019/020831, WO2020/157177, WO2020/157184 and EP21157154.2.

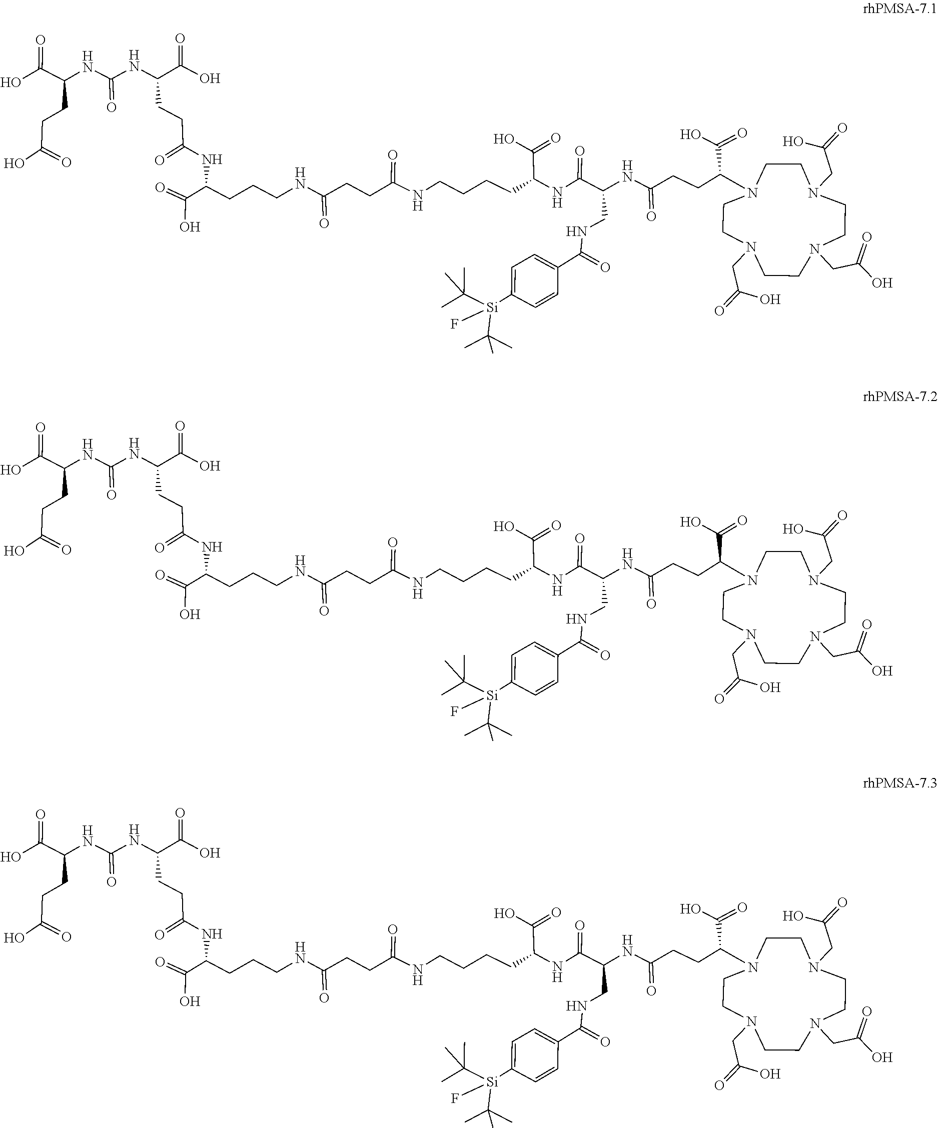

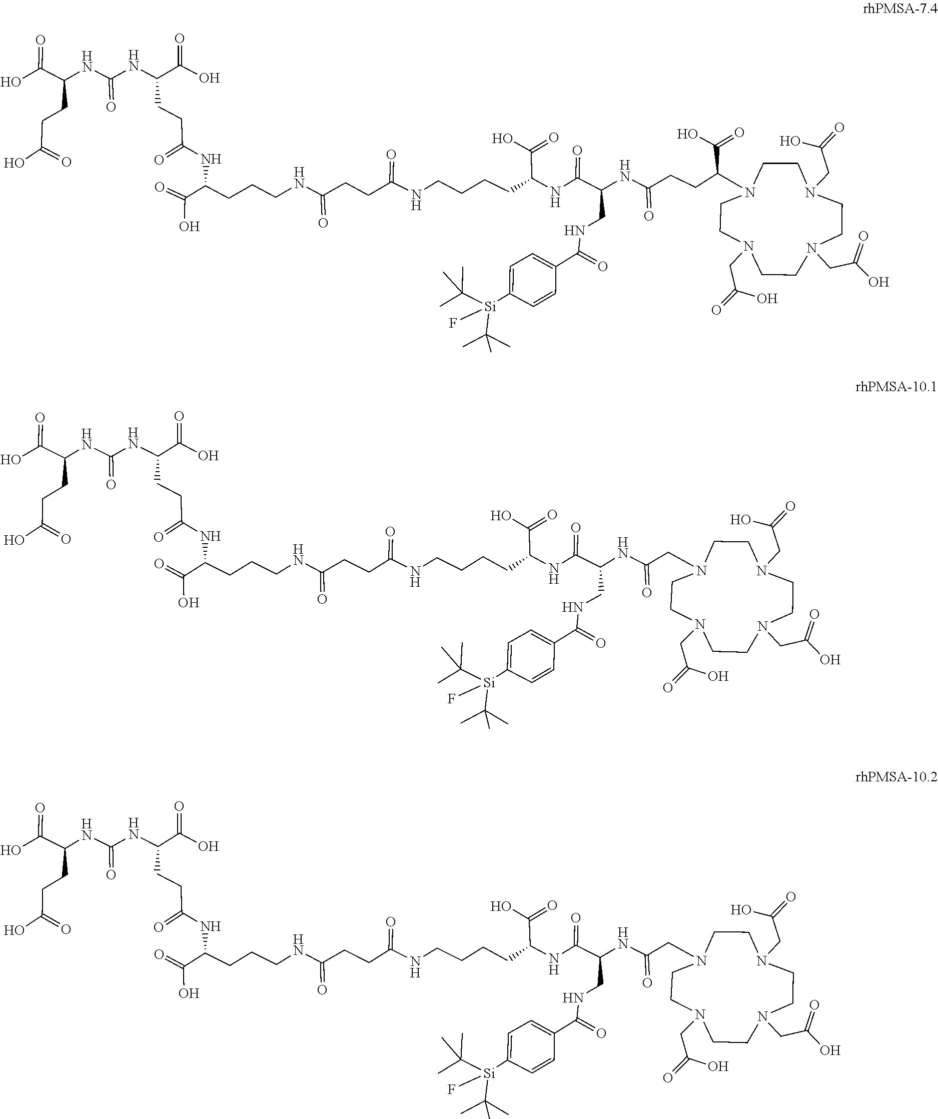

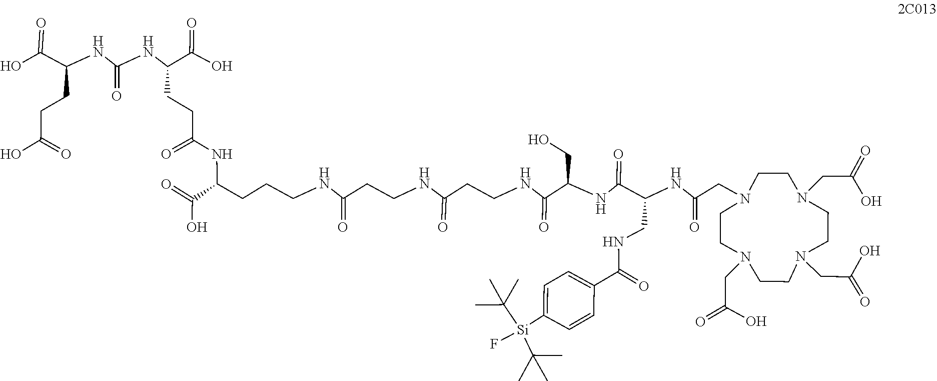 [SMILES: CC(C)(C)[Si](F)(c1ccc(C(=O)NC[C@@H](NC(=O)CN2CCN(CC(=O)O)CCN(CC(=O)O)CCN(CC(=O)O)CC2)C(=O)N[C@H](CO)C(=O)NCCC(=O)NCCC(=O)NCCC[C@@H](NC(=O)CC[C@H](NC(=O)N[C@@H](CCC(=O)O)C(=O)O)C(=O)O)C(=O)O)cc1)C(C)(C)C]

### ¹⁸F-Fluorination of rhPSMA-7.3

Aqueous ¹⁸F-was passed through a quaternary methyl ammonium carbonate anion exchange cartridge (Sep-Pak Accell Plus QMA Carbonate), which was preconditioned with 5 mL of water. ¹⁸F-was eluted with a 15 mg/mL cryptand 222 and 2.0 mg/mL potassium carbonate solution in acetonitrile/water (9/1 v/v). The resulting [¹⁸F]fluoride, cryptand and potassium carbonate solution was then azeotropically dried by heating at approx. 100° C. Before radiolabelling, a 160 mM solution of acetic acid in DMSO was used to dissolve 0.27 μmol of rhPSMA-7.3. The resulting rhPSMA-7.3 solution was added to azeotropically-dried [¹⁸F]fluoride and the reaction mixture was incubated for 5 minutes at room temperature. For purification, a solid-phase extraction cartridge containing a hydrophobic resin (Sep-Pak Plus Short tC18 cartridge), preconditioned with 5 mL EtOH, followed by 10 mL of H₂O was used. The labelling mixture was diluted with 5 mL citrate buffer (pH 5) and passed through the cartridge followed by 24 mL of citrate buffer. The ¹⁸F-rhPSMA-7.3 was eluted with 3 mL of a 1:1 mixture (v/v) of EtOH in water.

Previously the process made use of oxalic acid and the impact of oxalic acid content, with on-cartridge drying of alkaline [¹⁸F]fluoride/K222, on radionuclide incorporation with rhPSMA-7.3and similar silicon-fluorine acceptors (Kostikov, A. P. et al. Bioconjugate Chem. 2012, 23, 106-114) was evaluated. Maximum ¹⁸F-radiolabelling was reached when using approx. 30 μmol oxalic acid for 90 μmol of potassium hydroxide (acid-base molar ratio ˜0.6:1) (Wurzer, A. et al. EJNMMI radiopharm. chem. 6, 4 (2021)).

Although used in a limited quantity, oxalic acid may be toxic. Hence further development was conducted to replace oxalic acid with acetic acid, a common excipient for parenteral administration. Therefore, oxalic acid (dicarboxylic acid, 30 μmol) was replaced with 2 molar equivalents of acetic acid (monocarboxylic acid 60 μmol) and was shown to yield ¹⁸F-rhPSMA-7.3successfully using the Scintomics GRP synthesis module.

Implementation of the process with azeotropic drying of [¹⁸F]fluoride requires inverse addition, i.e., addition of acidified precursor solution to alkaline [¹⁸F]fluoride/K222 instead of addition of alkaline [¹⁸F] fluoride/K222 to the acidic precursor solution. As shown in FIG. 1, a higher amount of acid was required to prevent isomerisation of ¹⁸F-rhPSMA-7.3 or ¹⁹F-rhPSMA-7.3 in the presence of carbonate to related Compound A shown below. A decrease of radiolabelling conversion was also observed with increasing acid content. The optimised acetic acid amount for each process is provided in Table 1.

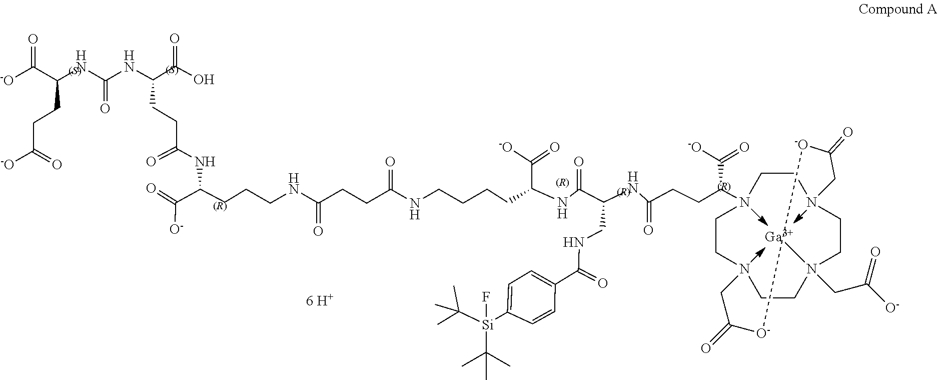

TABLE 1
Nominal acetic acid amounts for ¹⁸F-radiolabelling

| | | Azeotropic drying |
|---|---|---|
| Component | On-cartridge drying | process |
| Potassium hydroxide | 5.6 mg (100 μmol) | N/A |
| Potassium carbonate (K₂CO₃) | N/A | 2.8-7 mg (20-52 |
| | | μmol) |
| Acetic acid (AcOH) | 3.4 μL | 8.5-15 μL |
| | (60 μmol) | (150-260 μmol) |
| Molar ratio of AcOH/base | 0.6 | 2.9-7.7 |

### BRIEF DESCRIPTION OF THE FIGURES

FIG. 1: Impact of acetic acid content on isomerisation (formation of Related Compound A) and yield.

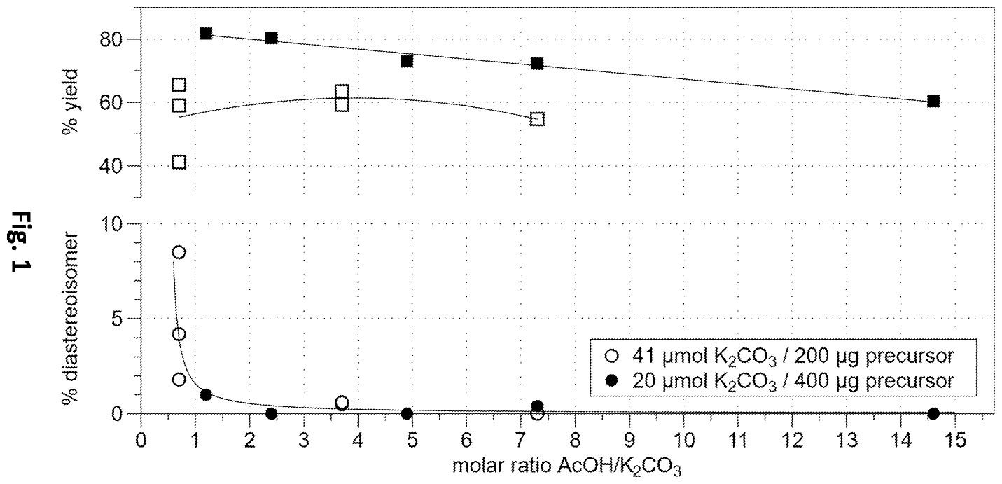

1. A method of preparing a solution comprising an ¹⁸F labelled silyl-fluoride compound, wherein the method comprises:
a) passing aqueous ¹⁸F solution through an anion exchange cartridge;
b) eluting the ¹⁸F from the cartridge using a solution comprising [2.2.2]-cryptand, an inorganic base, an organic solvent and water;
c) azeotropically drying the eluent; and
d) adding to the azeotropically dried eluent a solution comprising acetic acid and a compound having a ¹⁹F silyl-fluoride bond;
wherein said solution comprising the ¹⁸F labelled silyl-fluoride compound further comprises acetic acid at a concentration of at least 100 mM.

2. The method according to claim 1 wherein the solution comprising the ¹⁸F labelled silyl-fluoride compound comprises non-acetate organic acid species at a concentration of less than 0.3 mM.

3. The method according to claim 1 or claim 2, wherein the prepared solution comprising acetic acid and a compound having a ¹⁹F silyl-fluoride bond comprises an aprotic solvent.

4. The method according to claim 3, wherein the aprotic solvent is DMSO, acetonitrile or DMF, or any combination thereof.

5. The method according to any one of claims 1 to 4, wherein the inorganic base is potassium carbonate

6. The method according to any one of claims 1 to 5, wherein the organic solvent is acetonitrile.

7. The method according to any one of claims 1 to 6, wherein the acetic acid concentration in the solution comprising an ¹⁸F labelled silyl-fluoride compound is 100-200 mM.

8. The method according to any one of claims 1 to 6, wherein the acetic acid concentration in the solution comprising an ¹⁸F labelled silyl-fluoride compound is 130-160 mM.

9. The method according to any one of claims 1 to 8, wherein:
the ¹⁸F labelled silyl-fluoride compound is a compound of formula (1a): 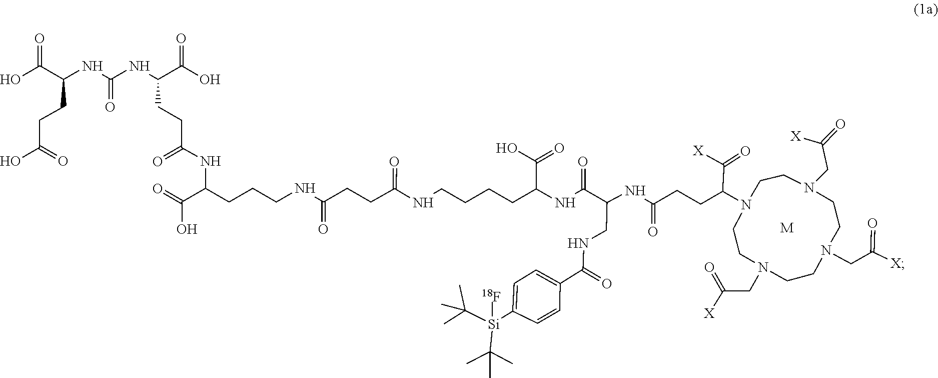
or the ¹⁸F labelled silyl-fluoride is a compound of formula (1b): 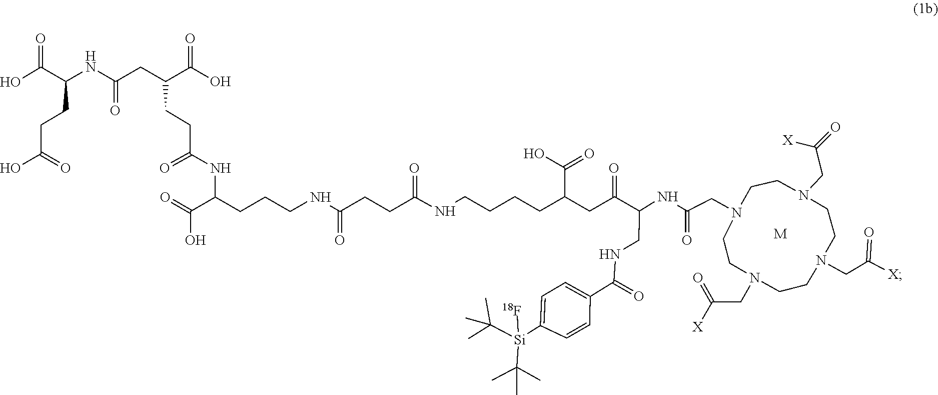
or the ¹⁸F labelled silyl-fluoride compound is a compound of formula (1c): 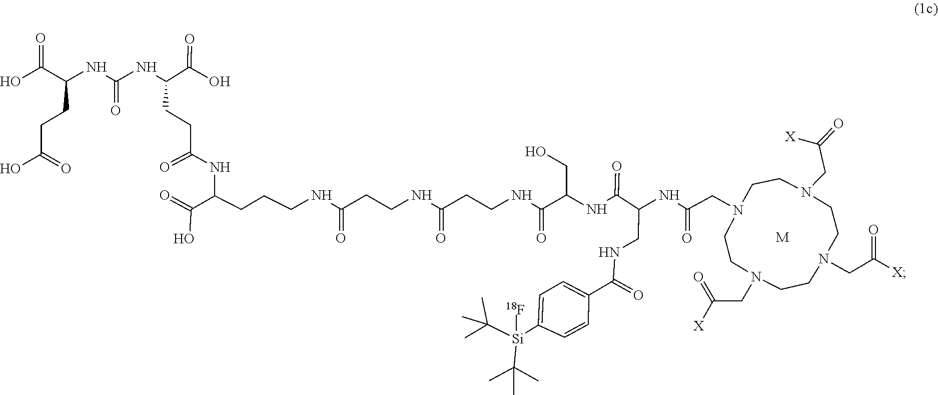
wherein each X is independently OH or O⁻; and
M is either a chelated cation or is absent.

10. The method according to any one of claims 1 to 8, wherein:
the ¹⁸F labelled silyl-fluoride compound is a compound of formula (3a): 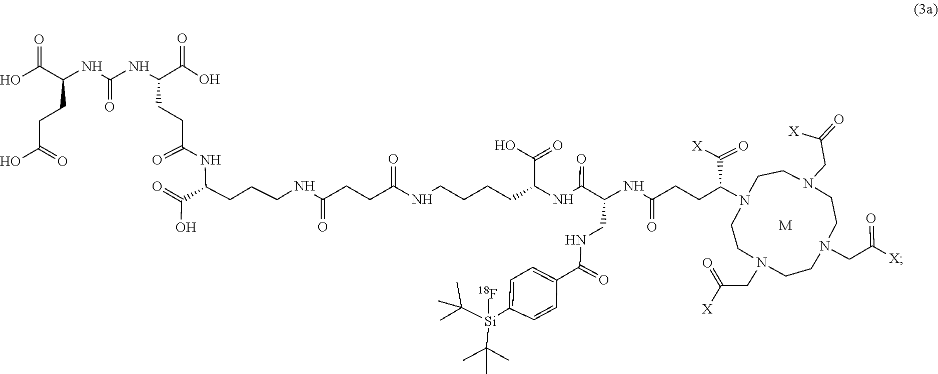
or the ¹⁸F labelled silyl-fluoride compound is a compound of formula (3b): 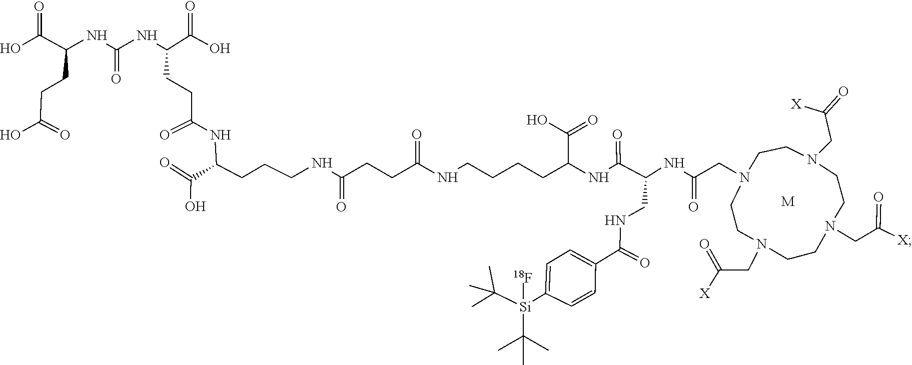
or the ¹⁸F labelled silyl-fluoride compound is a compound of formula (3c): 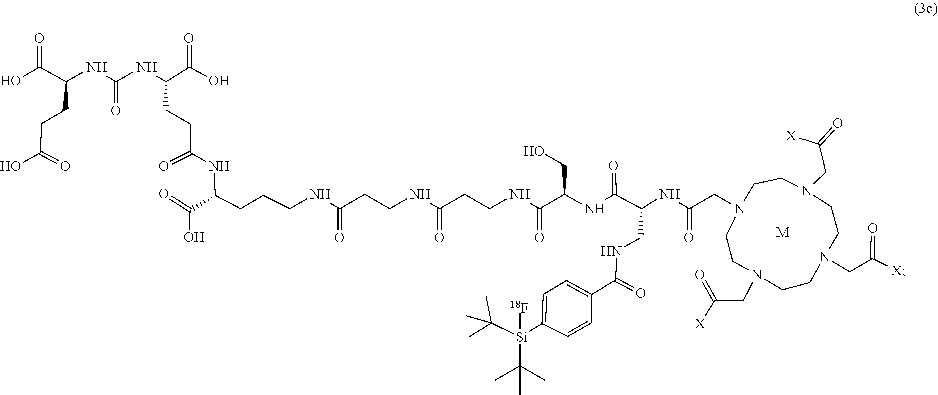
wherein each X is independently OH or O⁻; and
M is either a chelated non-radioactive cation or is absent.

11. The method according to claim 9 or claim 10, wherein M is selected from the cations of Sc, Cu, Ga, Y, In, Tb, Ho, Lu, Re, Pb, Bi, Ac, Er and Th.

12. The method according to claim 11, wherein M is Ga³⁺.

13. The method according to any one of claims 1 to 12, wherein:
the ¹⁸F labelled silyl-fluoride compound is a compound of formula (5a): 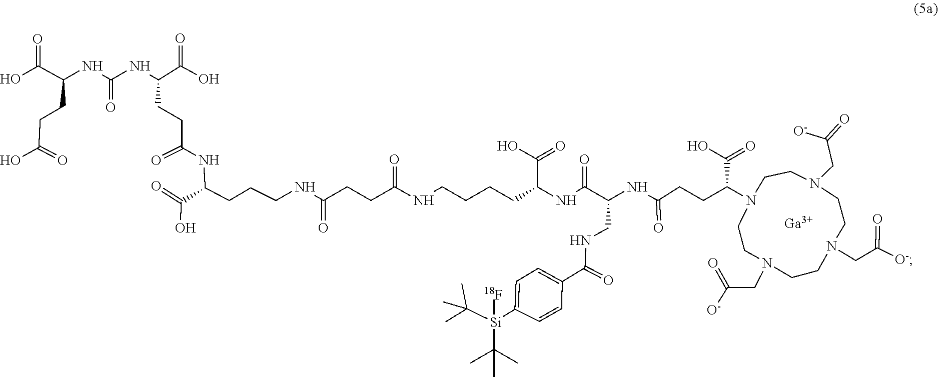
and the compound having a ¹⁹F silyl-fluoride bond is a compound of formula (6a):

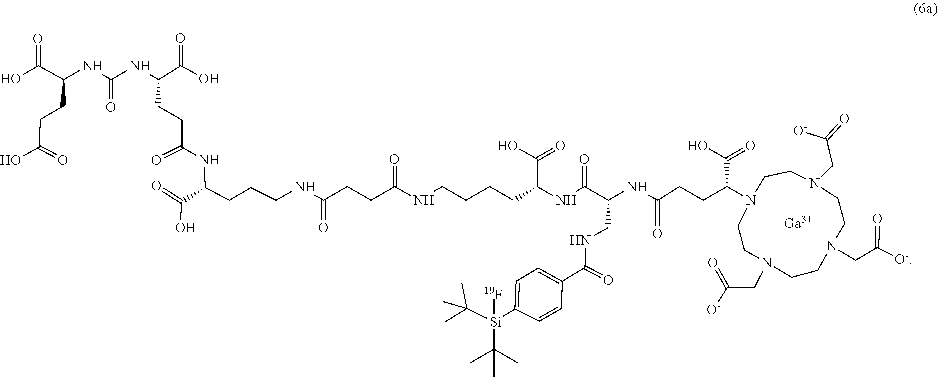

14. The method according to any one of claims 1 to 13, wherein the anion exchange cartridge is a quaternary methyl ammonium carbonate anion exchange cartridge.

15. The method according to any one of claims 1 to 14, wherein the anion exchange cartridge is preconditioned with water prior to addition of aqueous ¹⁸F solution.

16. The method according to any one of claims 1 to 15, wherein the solution comprising [2.2.2]-cryptand, an inorganic base, an organic solvent and water comprises:
2-10 mg/mL potassium carbonate and 15-53 mg/mL [2.2.2]-cryptand.

17. The method according to any one of claims 1 to 16, wherein the eluent is azeotropically dried with acetonitrile.

18. The method according to any one of claims 1 to 17, wherein the solution comprising acetic acid and a compound having a ¹⁹F silyl-fluoride bond in step d) is prepared using a 160mM solution of acetic acid in DMSO.

19. A liquid composition comprising an ¹⁸F labelled silyl-fluoride compound, acetic acid at a concentration of 100-200 mM and no non-acetate organic acid species at a concentration of 0.3 mM or higher.

20. The composition according to claim 19, wherein the ¹⁸F labelled silyl-fluoride compound is a compound of formula (1a), (1b) or (1c): 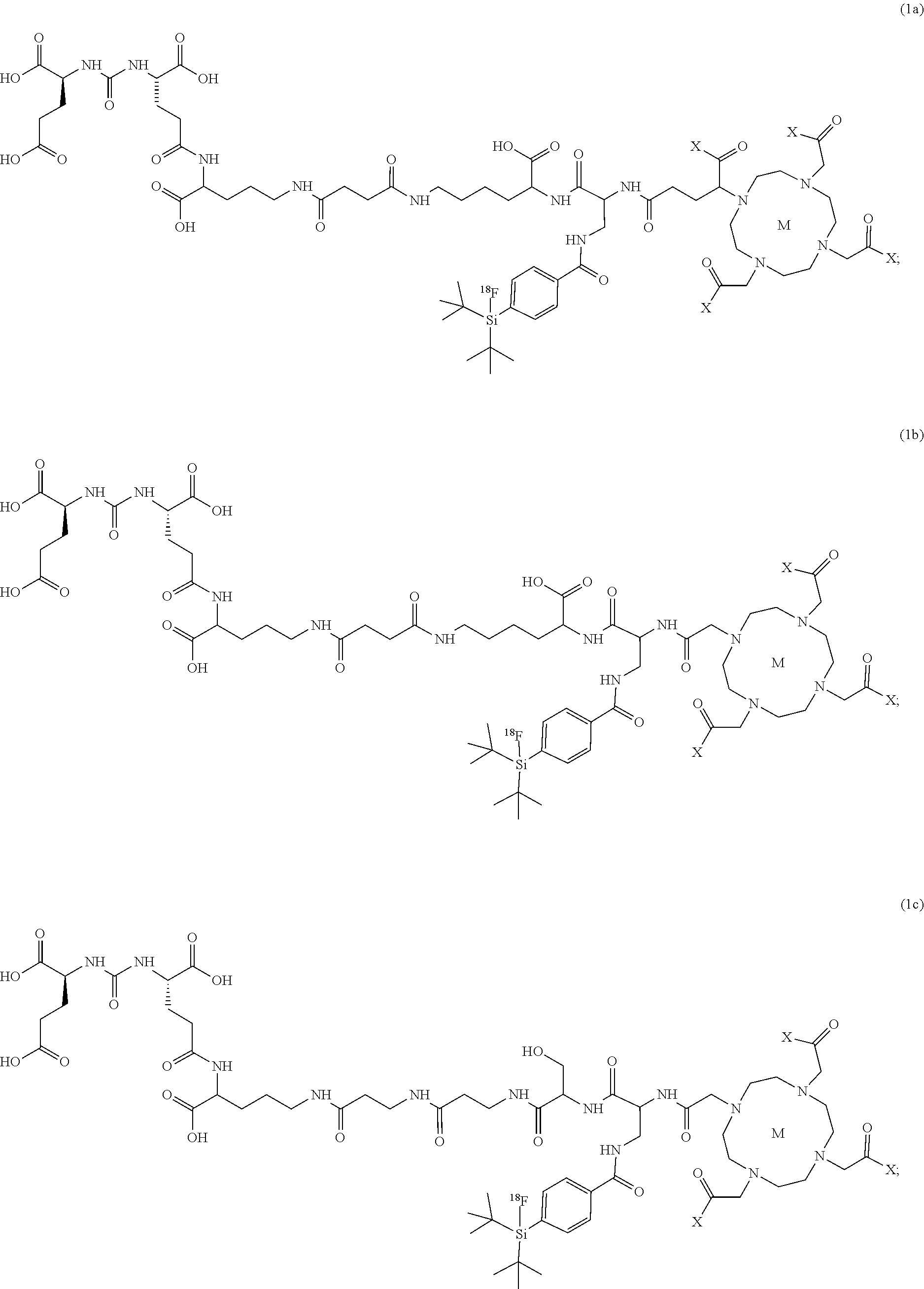
wherein each X is independently OH or O⁻; and
M is either a chelated cation or is absent.

21. The composition according to claim 19, wherein the ¹⁸F labelled silyl-fluoride compound is a compound of formula (3a), (3b) or (3c): 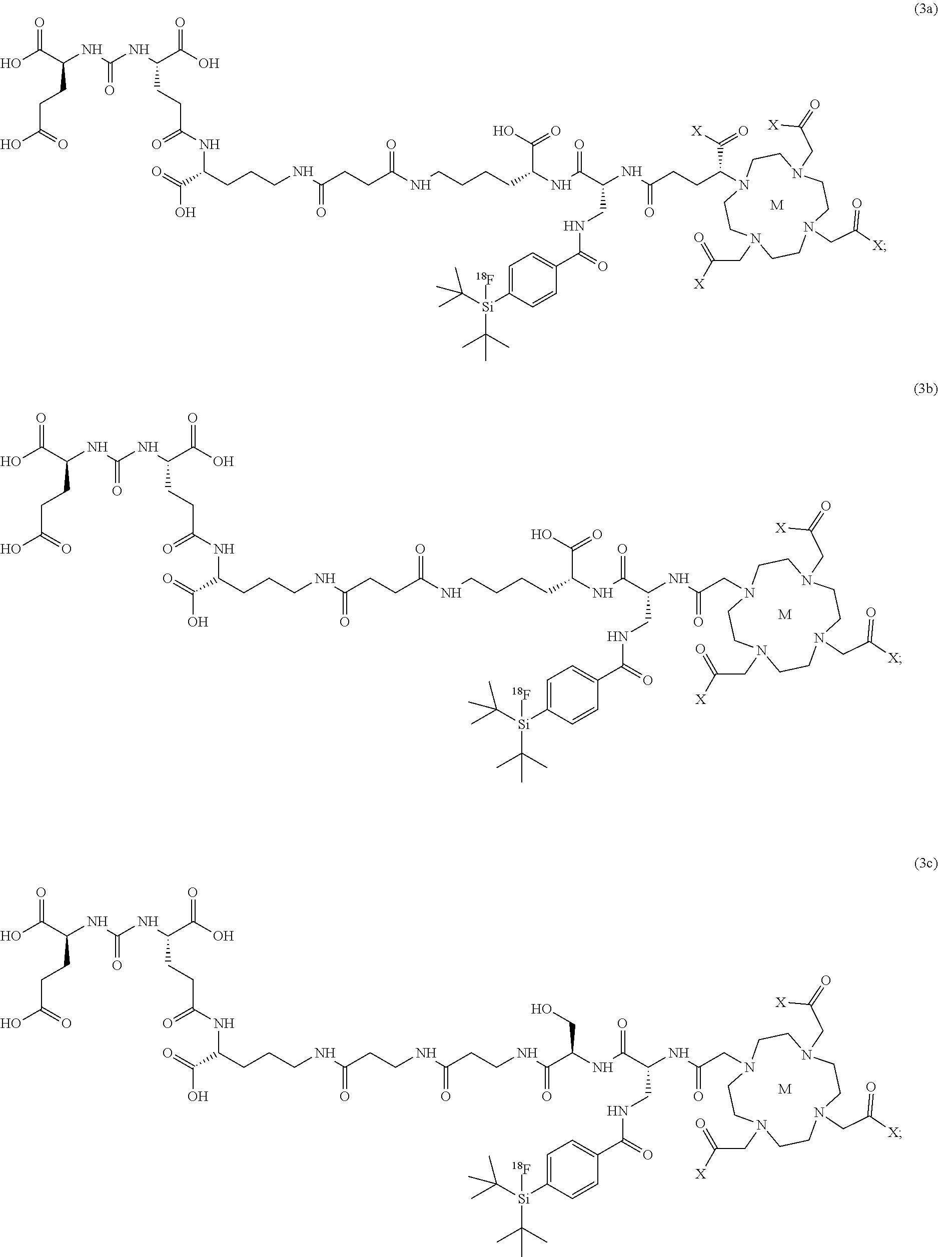
wherein each X is independently OH or O⁻; and
M is either a chelated cation or is absent.

22. The composition according to claim 19, wherein the ¹⁸F labelled silyl-fluoride compound is a compound of formula (5a):

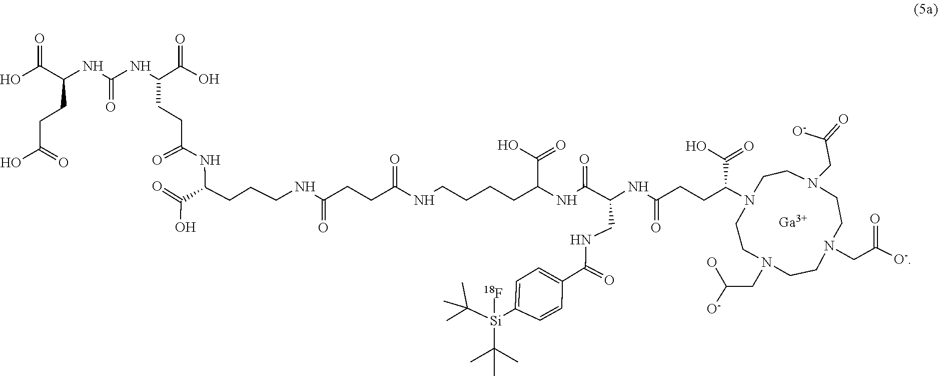

23. A composition prepared by a method comprising the method of any one of claims 1 to 19, for use in the diagnosis or imaging of neoangiogenesis/angiogenesis.

24. A composition prepared by a method comprising the method of any one of claims 1 to 19, for use as a cancer diagnostic or imaging agent wherein the cancer is prostate, breast, lung, colorectal or renal cell carcinoma.
